(19) United States
(12) Patent Application Publication
(10) Pub. No.: US 2023/0099264 A1
(43) Pub. Date: Mar. 30, 2023

(54) Biomarker for Predicting Prognosis of Radiotherapy for Lung Cancer

(71) Applicant: Korea Institute of Radiological & Medical Sciences, Seoul (KR)

(72) Inventors: Jong Kuk Park, Seoul (KR); Hong Duck Um, Seoul (KR); Sang Gu Hwang, Seoul (KR); Jie Young Song, Seoul (KR); Jeong Hyun Cho, Gwangmyeong-si, Gyeonggi-do (KR)

(21) Appl. No.: 17/800,728

(22) PCT Filed: Feb. 17, 2021
(86) PCT No.: PCT/KR2021/002039

§ 371 Date: Aug. 18, 2022

(30) Foreign Application Priority Data
Feb. 19, 2020 (KR) 10-2020-0020567

(51) Int. Cl. G01N 33/574

(52) U.S. Cl. CPC G01N 33/57423; G01N 2333/912; G01N 2333/545; G01N 2333/7155; G01N 2333/71; G01N 2333/82

(57) ABSTRACT

The present invention provides a method for providing information for diagnosis of metastasis of radiotherapy-treated lung cancer, the method comprising the steps of: (a) measuring an expression level of receptor-interacting protein kinase 1 (RIP1) in a sample from a lung cancer patient who has undergone radiotherapy; (b) measuring an expression level of RIP1 in a normal control sample; and (c) comparing the expression levels of step (a) and step (b).

### TECHNICAL FIELD

The present invention relates to a method for providing information for diagnosis of metastasis of radiotherapy-treated lung cancer, and more particularly, to a method for providing information for diagnosis of metastasis of radiotherapy-treated lung cancer using the expression level of Receptor-interacting protein kinase 1 (RIP1).

### BACKGROUND ART

Lung cancer is a type of cancer in developed countries, with the biggest cause being smoking and pollution. Lung cancer began to increase rapidly in countries in Europe and America at the turn of the 20th century, causing more than 1.3 million deaths worldwide every year. Lung cancer accounts for the highest proportion of cancer-related deaths. In Korea, it has been reported that about 100,000 new cancer patients are diagnosed every year, and about 50,000 cancer patients die every year. Moreover, the incidence of cancer has been increasing in recent years, and cancer is currently the second leading cause of death among adults in Korea. In particular, lung cancer accounts for about 12% of cancers occurring in Korean adults and ranks third in incidence after gastric cancer and liver cancer. In addition, the incidence of lung cancer is increasing in both men and women every year. In addition, lung cancer already shows metastasis to other organs when diagnosed as lung cancer or progresses locally even if there is no metastasis. Thus, despite various treatments such as radical resection, chemotherapy, and radiation therapy, it is a tumor with a very low cure rate, with a 5-year survival rate of about 5% due to recurrence and metastasis after treatment. In addition, since lung cancer occupies the first place in mortality due to cancer, there is a need to develop a treatment/therapeutic agent capable of minimizing side effects and increasing the treatment rate.

Currently, radiotherapy is an effective cancer treatment along with surgery or chemical drug therapy and is used for the purpose of killing cancer cells by treating cancer tissue in a patient with radiation.

However, although most cancer cells are removed after radiotherapy, the remaining cancer cells may acquire resistance to radiation in some cases. In this case, it is reported to be a major cause of difficulty in treating cancer patients by inducing cancer recurrence and metastasis.

Under this background, there is a need to discover cancer metastasis factors that increase after radiation treatment to cancer cells and to develop a therapeutic agent/therapeutic method that inhibits the same.

### DISCLOSURE

### Technical Problem

In the present invention, the expression of RIP1 is increased in lung cancer by radiotherapy, which confirms to increase invasion and migration in lung cancer.

Accordingly, one object of the present invention is to provide a method for providing information for diagnosing lung cancer metastasis by radiotherapy.

Another object of the present invention is to provide a kit for diagnosing lung cancer metastasis induced by radiotherapy, which includes an agent for measuring the expression level of RIP1.

### Technical Solution

The present invention is described in detail as follows. Meanwhile, each description and embodiment disclosed in the present invention may be applied to each other description and embodiment. That is, all combinations of the various elements disclosed herein fall within the scope of the present invention. In addition, it cannot be considered that the scope of the present invention is limited by the specific descriptions described below.

All terms (including technical and scientific terms) used in this specification may be used with meanings that can be commonly understood by those of ordinary skill in the art to which the present invention belongs. In addition, terms defined in a commonly used dictionary are not to be interpreted ideally or excessively unless clearly defined in particular.

Hereinafter, a method for providing information for diagnosing radiotherapy-treated lung cancer metastasis according to the present invention will be described in detail.

RIP1 is considered to be a kinase that plays an important role in determining cell life and death. RIP1 has a total of three domains: a serine/threonine kinase domain required for necroptosis, an intermediate domain containing a homozygous interaction motif, and a death domain for apoptotic activity. RIP1 ubiquitination promotes cell survival, and diubiquitination, on the other hand, causes apoptosis in the cell. Since several domains exist in RIP1, activation of RIP1 can lead to various results, such as NF-κB, mitogen-activated protein kinase (MAPK), apoptosis, or necroptosis. Necrostatin-1 (nec-1) blocks RIP1 kinase activity to prevent necroptosis induced by apoptosis. RIP3 regulates programmed necroptosis and increases RIP1 recruitment in the necrosome. In addition to RIP1 and RIP3 kinases, several other kinases are also involved in the phosphorylation of RIP1 and RIP3.

The present invention presumes that RIP1 is a candidate as a radiation-induced protein induced by radiation in lung cancer, and it is identified that RIP1 has a function of increasing the invasion and migration of lung cancer cells by radiotherapy. In addition, it is confirmed that proteins such as NF-κKB, STAT-3, Src, and IL-1β are activated in response to radiation.

As shown in FIG. 1, proteins including RIP1 induce EMT through the EGFR/NF-κB-RIP1-Src-STATS-IL-1β signaling pathway by irradiation, and thus it is confirmed that activated EMT is a factor that promotes resistance such as migration and metastasis of cancer cells.

Accordingly, the present invention has completed the present invention by confirming the possibility of using RIP1 as a diagnostic biomarker since it is possible to diagnose the metastasis of lung cancer induced by radiotherapy using the extent of RIP1 expression.

One aspect of the present invention for achieving the above object provides a method for providing information for diagnosis of metastasis of radiotherapy-treated lung cancer and the method may include the following steps of:

(a) measuring an expression level of receptor-interacting protein kinase 1 (RIP1) in a sample from a lung cancer patient who has undergone radiotherapy;

(b) measuring an expression level of RIP1 in a normal control sample; and

(c) comparing the expression levels of step (a) and step (b).

In the method of providing information for diagnosis of metastasis of radiotherapy-treated lung cancer according to the present invention, the method may further include (d) determining that the metastatic potential of radiotherapy-treated lung cancer is high when the expression level of step (a) is equal to or greater than the expression level of step (b).

In the present invention, the “expression level of RIP1” means the protein expression level of RIP1 or the mRNA expression level of RIP1.

The “patient sample” may refer to a sample derived from a patient who has undergone radiotherapy (irradiation). For example, it refers to a sample capable of specifying overexpression of RIP1 as a biomarker for diagnosing lung cancer metastasis and may include a sample such as tissue cells, whole blood, serum, plasma, saliva, sputum, cerebrospinal fluid or urine of a patient. However, the present invention is not limited thereto.

In the present invention, the expression level of RIP1 in a normal control is compared with the expression level of RIP1 in a radiotherapy-treated lung cancer patient or a radiotherapy-treated early-stage lung cancer patient with mild symptoms to diagnose whether the lung cancer has metastasized to other parts of the body in a patient suspected of lung cancer metastasis induced by radiotherapy or an early-stage lung cancer patient with mild symptoms. That is, the expression level of RIP1 from a patient's sample is measured, and the expression level of RIP1 from a sample having a normal RIP1 expression level, such as a sample from a normal person or a radiotherapy-untreated lung cancer sample is measured, followed by the comparison of two levels. Then, if the expression level of RIP1 in the patient's sample is equal to or higher than that of the normal sample, lung cancer metastasis can be predicted.

As used herein, the terms “irradiation,” “radiotherapy,” “IR irradiation, handling, and/or treatment” refer to local treatment methods that damage DNA of malignant cells. Normal cells have a greater ability to repair this damage than tumor cells. Irradiation refers to a treatment using such a difference and includes a method of treatment using a conventional meaning radiation. Irradiation may be divided into radical radiotherapy, adjuvant radiotherapy, and palliative radiotherapy depending on the treatment purpose. Radiation is a treatment that uses high-energy radiation to kill cancer cells, but it affects not only cancer cells but also normal tissues around them. Therefore, side effects may occur according to the treatment. One of the biggest problems with these side effects is cancer metastasis.

The measurement of the expression level of RIP1 may be to measure the amount of protein or mRNA.

The measurement of the expression level of RIP1 may be to measure the protein expression using Western blotting, antigen-antibody reaction, enzyme linked immunosorbent assay (ELISA), radioimmunoassay, radial immunodiffusion method, Ouchterlony immunodiffusion, immunohistochemistry, rocket immunoelectrophoresis, tissue immunostaining, immunoprecipitation assay, complement fixation assay, FACS, protein chip, etc. Specifically, the measurement of the protein amount may be performed using an antigen-antibody reaction. Characterization by the antigen-antibody reaction is preferably performed by Western blotting, antigen-antibody reaction, enzyme linked immunosorbent assay (ELISA), radioimmunoassay, radial immunodiffusion method, Ouchterlony immunodiffusion, immunohistochemistry, rocket immunoelectrophoresis, tissue immunostaining, immunoprecipitation assay, complement fixation assay, FACS, or protein chip.

The measurement of the expression level of RIP1 may also be to measure the mRNA expression level by reverse transcription polymerase reaction (RT-PCR), competitive reverse transcription polymerase reaction (Competitive RT-PCR), real-time reverse transcription polymerase reaction (Real-time RT-PCR), RNase protection assay (RPA), Northern blotting, a DNA chip, etc.

The agent for measuring the mRNA expression level of a gene is preferably a primer pair or a probe, and since the nucleic acid information of the genes is known from GeneBank, etc., those skilled in the art can design a primer or probe that specifically amplifies a specific region of these genes based on the sequence.

In the present invention, the measurement of the expression level of steps (a) and (b) may further include the measurement of the expression level of any one or more selected from the group consisting of EGFR, NF-κB, STAT-3, Src, IL-1RI, IL-1RII, and IL-1β.

Specifically, the present invention may include steps of:

(a) further measuring the expression level of any one or more selected from the group consisting of EGFR, NF-κB, STAT-3, Src, IL-1RI, IL-1RII, and IL-1β from radiotherapy-treated lung cancer patient sample,

(b) further measuring the expression level of any one or more selected from the group consisting of EGFR, NF-κB, STAT-3, Src, IL-1RI, IL-1RII, and IL-1β from the normal control sample, and

(c) comparing the expression levels of step (a) and step (b).

Preferably, the expression levels of all of the EGFR, STAT-3, Src, IL-1RI, IL-1RII, and IL-1β can be measured. In samples from lung cancer patients, the above-mentioned EGFR, NF-κB, STAT-3, Src, IL-1RI, IL-1RII, and IL-1β expressions all show an increase by radiotherapy compared to normal controls. Therefore, it is possible to more accurately determine the metastasis potential of lung cancer by radiotherapy by measuring the above-mentioned factors together with the measurement of RIP1.

Therefore, receptor-interacting protein kinase 1 (RIP1) can be used as a diagnostic biomarker for diagnosing radiation-induced lung cancer metastasis. Any one or more selected from the group consisting of EGFR, NF-κB, STAT-3, Src, IL-1RI, IL-1RII, and IL-1β may be further included as an additional diagnostic biomarker.

In the present invention, “biomarker” refers to a molecular marker for the purpose of predicting the diagnosis of lung cancer metastasis induced by radiation and is used interchangeably to refer to a target molecule that indicates or is a sign of a normal or abnormal process in an individual or of a disease or other condition in an individual. More specifically, it is an anatomic, physiologic, biochemical, or molecular parameter associated with the presence of a specific physiological state or process. A biomarker is detectable and measurable by a variety of methods including laboratory assays and medical imaging.

The above-mentioned measurement of the expression level of any one or more selected from the group consisting of EGFR, NF-κB, STAT-3, Src, IL-1RI, IL-1RII, and IL-1β may be to measure the amount of the aforementioned protein or mRNA.

Another aspect of the present invention for achieving the above object, the present invention provides a kit for diagnosing lung cancer metastasis induced by radiation, the kit including an agent for measuring the expression level of RIP1 (Receptor-interacting protein kinase 1). Preferably, the kit may be an RT-PCR kit, a DNA chip kit, an ELISA kit, a protein chip kit, a rapid kit, or a multiple reaction monitoring (MRM) kit. Preferably, the diagnostic kit may further include one or more other component compositions, solutions, or devices suitable for the assay method.

The kit may further include an agent for measuring the expression level of any one or more selected from the group consisting of EGFR, NF-κB, STAT-3, Src, IL-1RI, IL-1RII, and IL-1β.

For example, it may be a diagnostic kit characterized in that it includes essential elements necessary for performing an ELISA. The ELISA kit includes an antibody, an interacting protein, a ligand, nanoparticles, or an aptamer that specifically binds to the protein or peptide fragment. The antibodies may be monoclonal, polyclonal, or recombinant antibodies, which have high specificity and affinity to each marker protein and rarely have cross-reactivity to other proteins. Also, the ELISA kit may further include reagents capable of detecting bound antibodies, for example, a labeled secondary antibody, chromophores, enzymes (e. g., conjugated with an antibody) and other substances capable of binding to their substrates or the antibodies.

In addition, the kit may further include a user's instruction manual describing optimal conditions for performing the reaction. The instruction manual includes a pamphlet or leaflet-type brochure, labels attached to the kit and instructions on the surface of the package including kit.

In addition, the instruction manual may include information published or provided through an electronic medium such as the Internet.

The present invention provides a method for inhibiting metastasis of radiotherapy-treated lung cancer, which may include the following steps:

(a) measuring an expression level of receptor-interacting protein kinase 1 (RIP1) in a sample from a lung cancer patient who has undergone radiotherapy;

(b) measuring an expression level of RIP1 in a normal control sample;

(c) comparing the expression levels of step (a) and step (b); and

(d) when the expression level of step (a) is higher than the expression level of step (b), administering a therapeutic agent for lung cancer or a metastasis inhibitor in a therapeutically effective amount.

The individual for administering the therapeutic agent or the metastasis inhibitor is diagnosed with lung cancer and refers to a human or non-human mammal in need of treatment thereof.

The therapeutically effective amount may include (a) a decrease in the severity or persistence of a symptom or sign; (b) inhibition of tumor growth and metastasis, or an increase in tumor necrosis, tumor shrinkage and/or tumor disappearance; (c) delay in tumor growth and pathogenesis; (d) inhibition of tumor metastasis; (e) prevention of tumor growth recurrence, etc.

Preferably, the expression levels of all of the EGFR, NF-κB, STAT-3, Src, IL-1RI, IL-1RII, and IL-1β can be measured. In samples from lung cancer patients, the above-mentioned EGFR, NF-κB, STAT-3, Src, IL-1RI, IL-1RII, and IL-1β expressions all show an increase by radiotherapy compared to normal controls.

Therefore, it is possible to more accurately determine the metastasis potential of lung cancer by radiotherapy by measuring the above-mentioned factors together with the measurement of RIP1.

Such a lung cancer therapeutic agent or metastasis inhibitor may include, for example, a DNA alkylating agent, such as mechlorethamine, chlorambucil, phenylalanine, mustard, cyclophosphamide, ifosfamide, carmustine (BCNU), lomustine (CCNU), streptozotocin, busulfan, thiotepa, cisplatin, and carboplatin; anti-cancer antibiotics such as dactinomycin (actinomycin D), doxorubicin (adriamycin), daunorubicin, idarubicin, mitoxantrone, plicamycin, mitomycin, and C bleomycin; and plant alkaloids such as vincristine, vinblastine, paclitaxel, docetaxel, etoposide, teniposide, topotecan, and iridotecan, but is not limited thereto.

### Advantageous Effects

The present invention provides a method for providing information for diagnosing lung cancer metastasis by radiotherapy, thereby effectively predicting the occurrence of lung cancer metastasis to improve the survival rate of lung cancer patients.

In addition, it can be utilized as a specific target in the development of a specific therapeutic agent that can inhibit lung cancer metastasis induced by radiotherapy. Therefore, there is an effect that can provide accurate basic information for planning and performing an effective protocol of future treatment by a clinician.

### DESCRIPTION OF DRAWINGS

FIG. 1 is a schematic view illustrating a signaling system of cancer metastasis that occurs by ionizing radiation (IR) treatment in lung cancer.

FIGS. 2A-2D show the results of analyzing the invasion and migration of lung cancer cells by IR treatment.

FIGS. 3A-3D show the results of the analysis of signaling related to RIP1 expression and epithelial-mesenchymal transition (EMT) generation by IR treatment.

FIGS. 4A-4E show the results of confirming that cancer metastasis by IR treatment is inhibited when RIP1 expression is inhibited (necrostatin treatment).

FIGS. 5A-5D show the results of confirming that cancer metastasis by IR treatment is inhibited when RIP1 expression is inhibited (siRNA treatment for RIP1).

FIGS. 6A-6D show the results of confirming that cancer metastasis by IR treatment is inhibited when NF-κB is inhibited.

FIGS. 7A-7C show the results of confirming that cancer metastasis by IR treatment is inhibited and the expression of IL-1β is reduced when NF-κB is inhibited.

FIGS. 8A-8B show the results of confirming the effect of RIP1 on NF-κB activation mechanism and cell migration according to IL-1Ra treatment.

FIGS. 9A-9D show the results of confirming the expression change of IR-induced IL-1β and its receptor IL-lri/II.

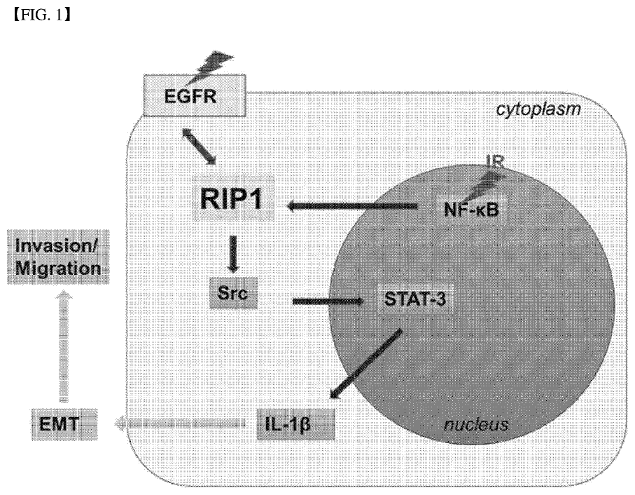

### MODES OF THE INVENTION

Hereinafter, examples will be described in detail to help the understanding of the present invention. However, the following examples are merely illustrative of the content of the present invention, and the scope of the present invention is not limited to the following examples. The examples of the present invention are provided to more completely explain the present invention to those of ordinary skill in the art.

### Example 1: Radiation (IR)—Induced Lung Cancer Cell Line Culture (A549 IR)

Human A549 lung cancer cells were cultured in RPMI-1640 medium supplemented with 10% heat inactivated fetal bovine serum and maintained at 37° C. under 5% CO₂.

Lung cancer cells (A549) (5×10⁵) were seeded in 60 mm dishes and cultured overnight, followed by IR exposure (10 Gy) using a 137 Cs radiation source (Atomic Energy of Canada, Ltd., Mississauga, ON, Canada) to obtain IR-treated lung cancer cell lines (A549 IR (10 Gy)).

In the following examples, experiments were performed using the cells cultured in Example 1.

### Example 2: Analysis of the Effect of RIP1 Expression on Migration and Invasion of Cancer Cells in Radiation-Induced Lung Cancer Cells

Invasion and migration analysis of the IR-treated lung cancer cells (A549 IR) obtained in Example 1 was performed, and an experiment was performed to confirm the factors expressed by IR.

In order to analyze the cell invasion of lung cancer cells by IR treatment, Matrigel-coated transwells (Corning N. Y., USA) were used, and in order to analyze cell migration, collagen-coated transwells (Corning, N.Y., USA) were used.

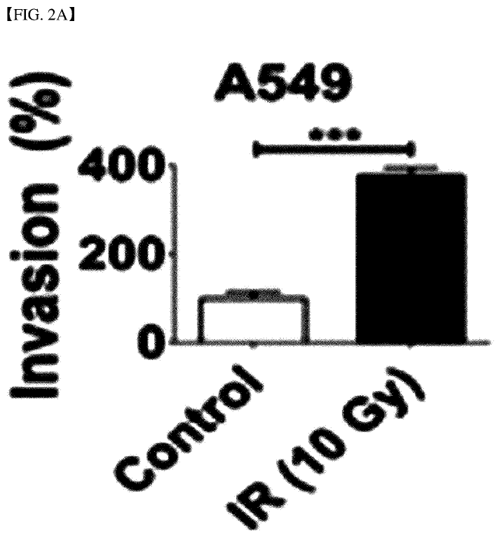

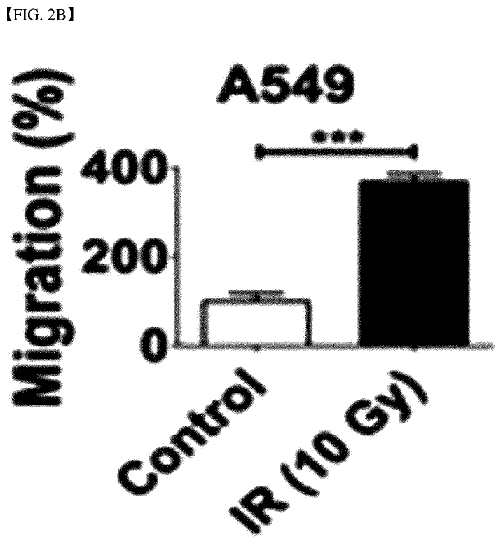

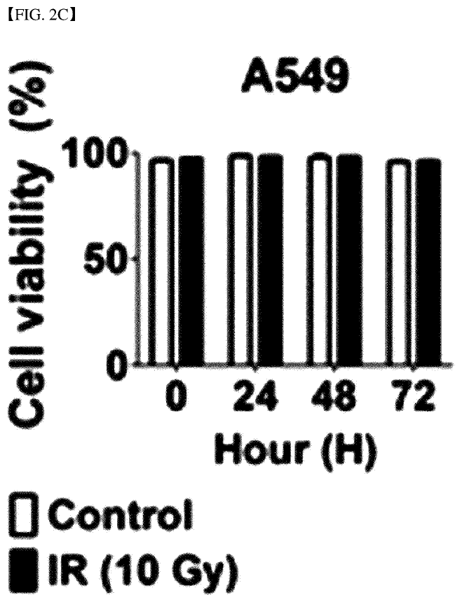

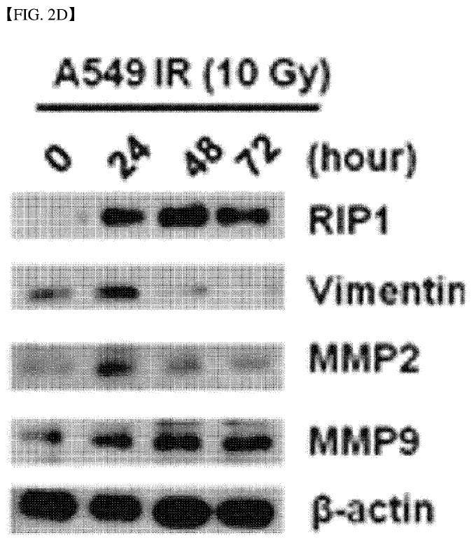

The IR-treated lung cancer cell line (A549 IR, 200 ul) obtained in Example 1 was seeded in the upper chamber by 1×10⁴ each in the medium of Matrigel-coated trenswell and collagen-coated transwell, and serum-free medium supplemented with 0.1% BSA (bovine serum) was placed in the lower chamber. Each transwell was cultured at 37° C. under 5% CO₂ for 18 hours. Thereafter, cell staining was performed with a deep quick solution (Merck, Whitehouse Station, N.J., USA) to confirm invasion and cell migration, and the results are shown in FIGS. 2A and 2B.

As shown in FIGS. 2A and 2B, it was confirmed that the cell invasion and migration of the IR-treated lung cancer cell line were increased. Meanwhile, as shown in FIG. 2C, the cell viability was confirmed to be almost similar to that of the lung cancer cell line that was not treated with radiation (control group).

In addition, for the analysis of RIP1 expression by IR treatment, the IR-treated lung cancer cell lines (A549 IR) cultured in Example 1 were seeded and harvested 24 hours later, and then protein was extracted. Western blot was performed to confirm the expressions of RIP1, vimentin, MMP2, MMP9, and β-action, and the results are shown in FIG. 2D.

As shown in FIG. 2D, treatment with IR in lung cancer cell lines increased the expression of RIP1 and increased the expressions of vimentin, MMP2, and MMP9.

Accordingly, it was confirmed that radiation treatment increased the expression of RIP1 in lung cancer cells and affected the metastasis and invasion ability of the cells and that radiotherapy could affect the metastasis of lung cancer, and RIP1 was identified as a related factor.

### Example 3: Analysis of RIP1 Expression and EMT Expression According to Radiation Treatment Using Lung Cancer Cell Line

In order to investigate the intracellular relationship between RIP1 and EMT, an analysis of RIP1 and EMT-related protein expressions was performed in lung cancer cell lines.

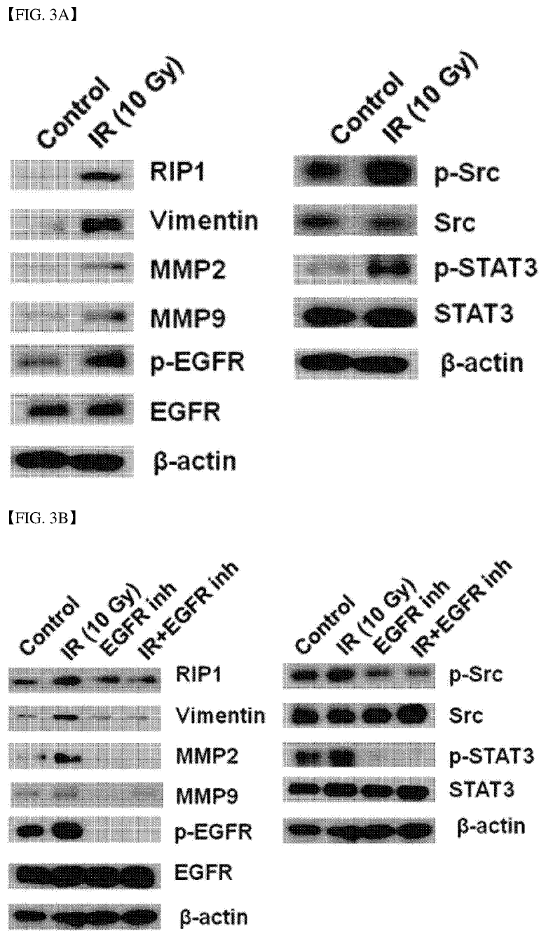

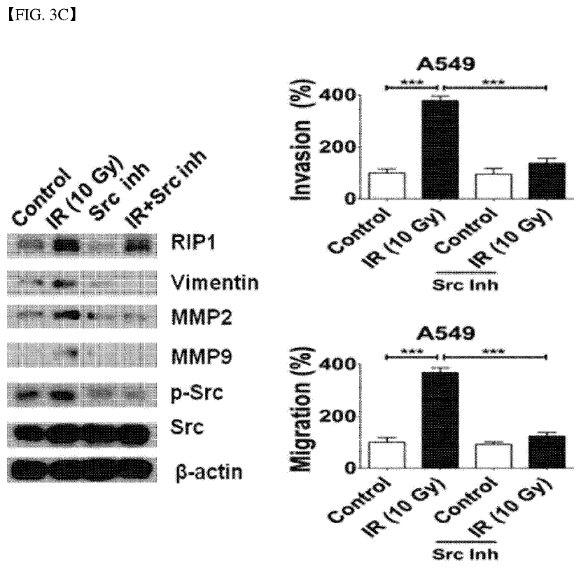

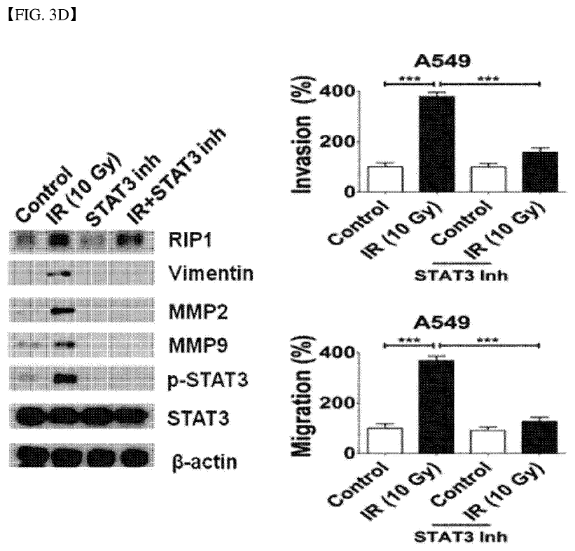

Each of A549 lung cancer cell line (control) and IR-treated A549 lung cancer cell line was seeded with 1×10⁶ in a 60π dish, and cells were harvested 24 hours later, and then protein was extracted. Western blot was performed to confirm the expressions of RIP1, vimentin, MMP2, MMP9, p-EGFR, EGFR, β-action, p-Src, Src, p-STAT3, and STATS, and the results are shown in FIG. 3A.

As shown in FIG. 3A, it was confirmed that the expressions of RIP1, vimentin, p-EGFR, and p-STAT3 were strong in the IR-treated lung cancer cell line compared to the control group, and even the expressions of p-Src, MMP2, and MMP9 were increased after IR irradiation.

The above results confirmed that the expression of EMT-related molecules was changed according to the expression of RIP1 by IR treatment, which indicated that RIP1 and EMT-related molecules had a correlation. The correlation between RIP1 and EMT-related molecules revealed that RIP1 could induce EMT generation.

### Example 4: Activation Analysis of EGFR, Scr, and STAT3 Signaling by IR

In the above Examples, it was confirmed that EGFR, Scr, and STAT3 signaling mechanisms were important signaling mechanisms among cell migration signaling mechanisms. EGFR, Scr, and STAT3 signaling mechanisms were identified. Thus, an experiment was performed to determine whether these EGFR, Scr, and STAT3 signaling were activated by IR.

Lung cancer cell line A549 (control) and IR-treated lung cancer cell line A549 (IR) were placed in an upper chamber by 5×10⁴ in a Matrigel-coated transwell, and treated with EGFR, Scr, and STAT3 inhibitors, respectively. Then, 10% FBS medium was put into the lower chamber, and cells passing through the transwell were stained after 24 hours to confirm cell invasion, and the results are shown in FIGS. 3C and 3D.

In addition, lung cancer cell line A549 (control) and IR-treated lung cancer cell line A549 (IR) were seeded at 1×10⁶ in a 60π dish and treated with EGFR inhibitor (EGFR inh), Src inhibitor (Src inh), and STAT3 inhibitor (STAT3 inh). The cells were treated by IR (IR-EGFR inh, IR-Src inh, and IR-STAT3 inh). The cells were harvested 24 hours later, and protein was extracted. Then, Western blot was performed to confirm the expressions of RIP1, vimentin, MMP2, MMP9, p-Src, Src, p-STAT3, STAT3, and β-action, and the results are shown in FIG. 3B (EGFR inhibitor), FIG. 3C (Src inhibitor), and FIG. 3D (STAT3 inhibitor).

Referring to the results of cell invasion and metastasis analysis shown in FIGS. 3C and 3D, it was confirmed that cell invasion and cell migration were inhibited when the IR-treated lung cancer cell line was treated with a Scr inhibitor or a STAT3 inhibitor.

In addition, as shown in FIGS. 3B, 3C, and 3D, Western blot was performed to observe RIP1 expression and signaling mechanisms when treatment of IR-treated EGFR inhibitor, STAT3 inhibitor, and Src inhibitor. As a result, it was confirmed that RIP1 expression was decreased during EGFR inhibition, STAT3 inhibition, and Src inhibition, and signaling of EGFR, Src, and STAT3 was decreased, respectively.

The above results reveal that phosphorylation of EGFR was induced by RIP1 expressed in the IR-treated lung cancer cell line, activation of a sub-molecule Src-STAT3 occurred by phosphorylation of EGFR, and cell migration was increased by induction of EMT.

### Example 5: Analysis of Signaling Mechanisms of RIP1 Affecting Cell Migration of IR-Treated Lung Cancer Cell Lines

Necrostatin, an inhibitor of RIP1 kinase, was used to inhibit function of RIP1 kinase, and then changes in EMT activation of IR-treated lung cancer cell lines were observed.

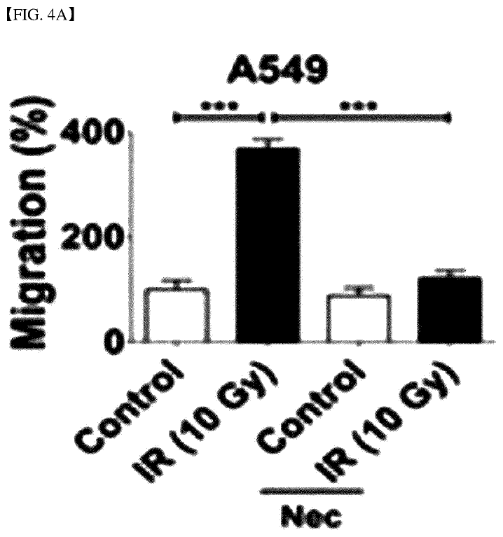

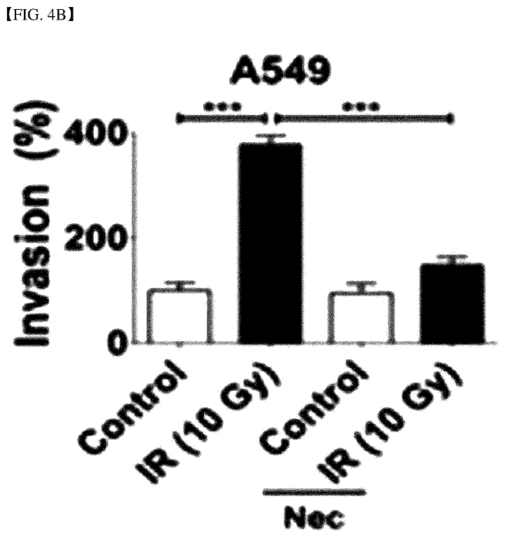

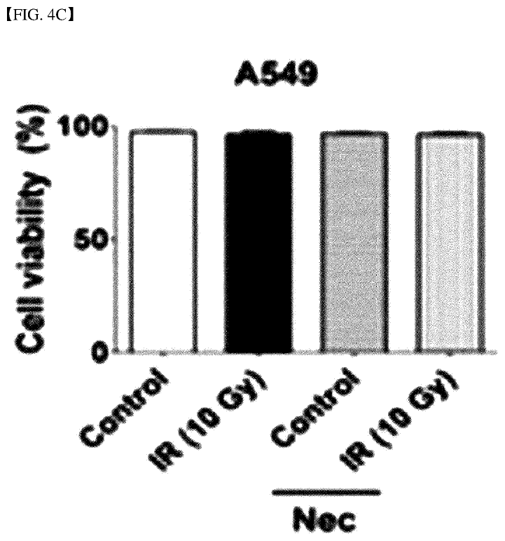

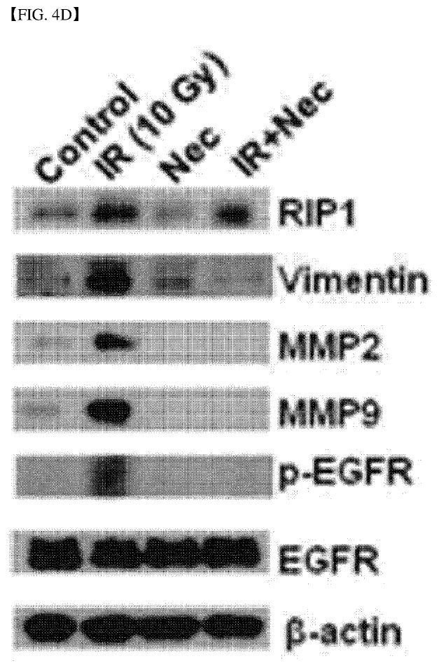

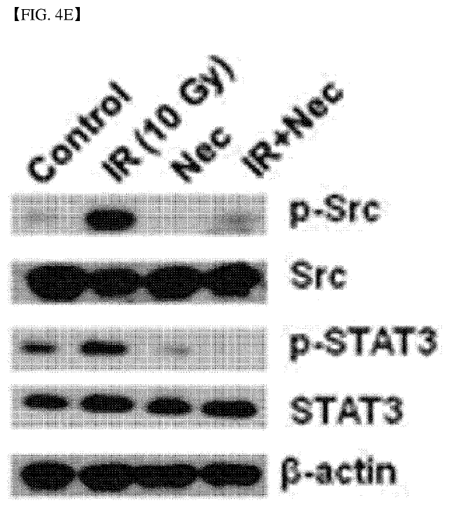

Lung cancer cell line A549 (control) and IR-treated lung cancer cell line A549 (IR) were placed in an upper chamber by 5×10⁴ in a Matrigel-coated transwell and treated with necrostatin. Then, 10% FBS medium was put into the lower chamber, and cells passing through the transwell were stained after 24 hours to confirm cell invasion, and the results are shown in FIGS. 4A and 4B.

As shown in FIGS. 4A and 4B, after treatment with necrostatin, a kinase inhibitor of RIP1, invasion and migration assays were performed to observe cell invasion and cell migration. As a result, it was confirmed that invasion and migration in IR-treated lung cancer cell lines was reduced by necrostatin treatment. Meanwhile, as shown in FIG. 4C, it was confirmed that the cell viability was almost similar to that of the lung cancer cell line (control) that was not treated with radiation.

In addition, lung cancer cell line A549 (control) and IR-treated lung cancer cell line A549 (IR) were seeded at 1×10⁶ in a 60π dish and treated with necrostatin. The cells were harvested 24 hours later, and protein was extracted. Then, Western blot was performed to confirm the expressions of RIP1, vimentin, MMP2, MMP9, p-EGFR, EGFR, β-action, p-Src, Src, p-STAT3, and STAT3, and the results are shown in FIGS. 4D and 4E.

In addition, as shown in FIGS. 4D and 4E, RIP1 expression upon inhibition of RIP1 kinase was observed by performing Western blot. It was confirmed that inhibition of kinase effect leaded to decreased expression of RIP1, and decreased activities of vimentin, MMP2, MMP9, p-EGFR, p-Src, and p-STAT3.

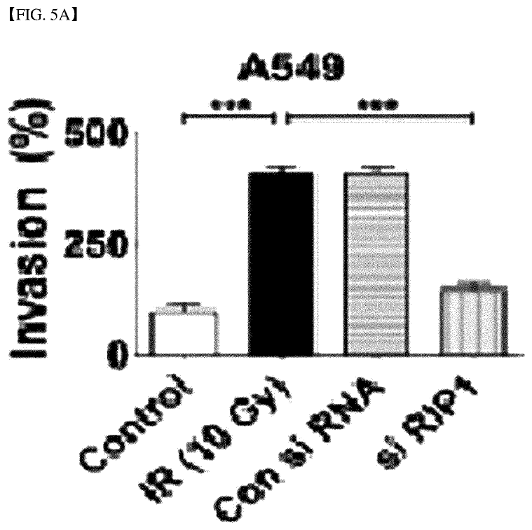

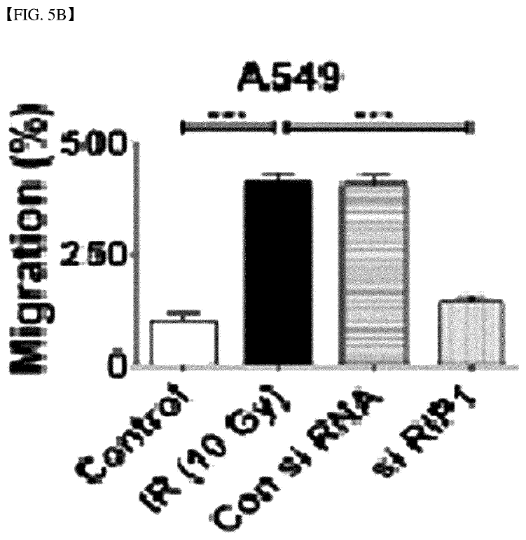

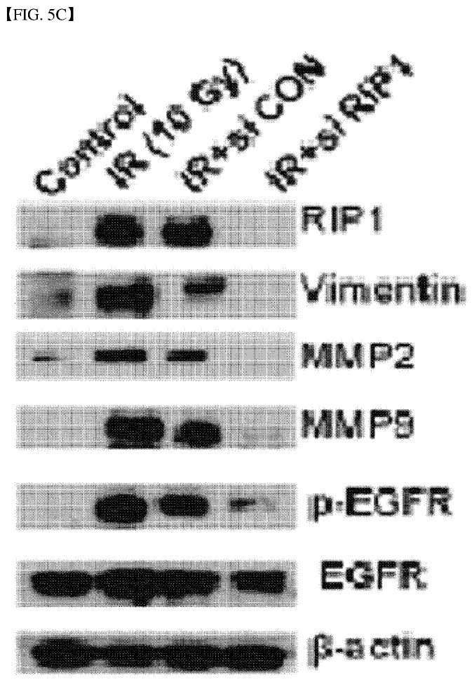

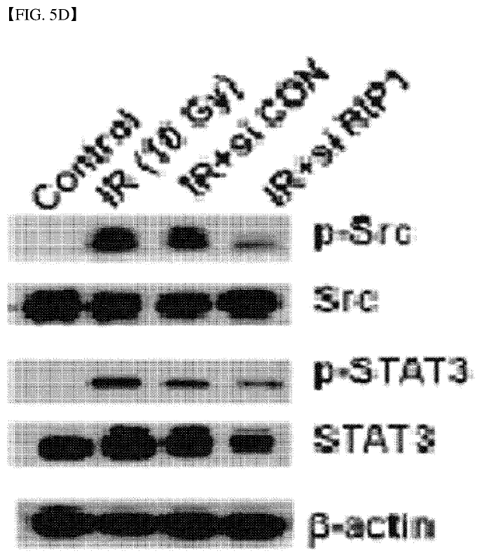

In addition, the same experiment as the above experiment was performed by treating a commercially available siRNA capable of inhibiting the expression of RIP1, and the results are shown in FIGS. 5A-5D.

As shown in FIGS. 5A and 5B, siRNA treatment of RIP1 significantly reduced the invasive and metastatic capacity of cells. In addition, as shown in FIGS. 5C and 5D, siRNA treatment also reduced the expression of EMT-related factors.

The above results confirm that cancer cell migration was increased by EMT activity by RIP1 in IR-treated lung cancer cell lines. In addition, it was shown that inhibition of RIP1 could inhibit EMT activity and reduced cell invasion and metastasis ability, thereby preventing and treating lung cancer metastasis.

### Example 6: Effects of RIP1 on NF-κB Activation Mechanism and Cell Migration in IR-Treated Lung Cancer Cell Lines

An experiment was conducted on whether RIP1 affects the activation mechanism of nuclear factor kappa-light-chain-enhancer of activated Bcells (NF-κB) and cell migration.

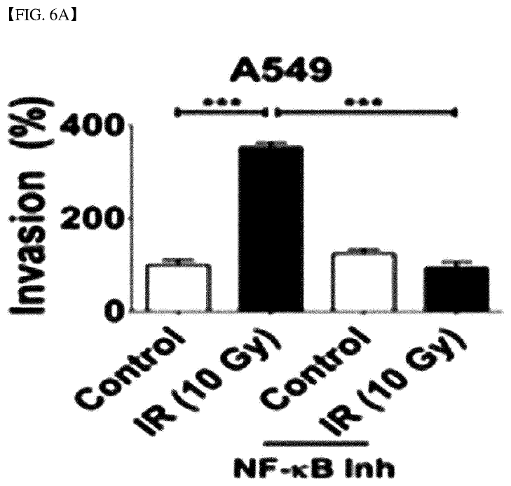

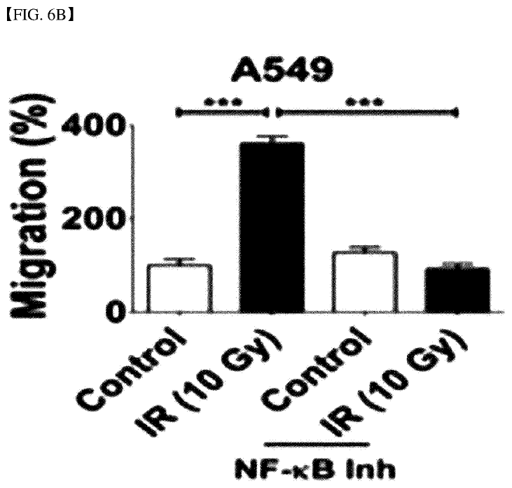

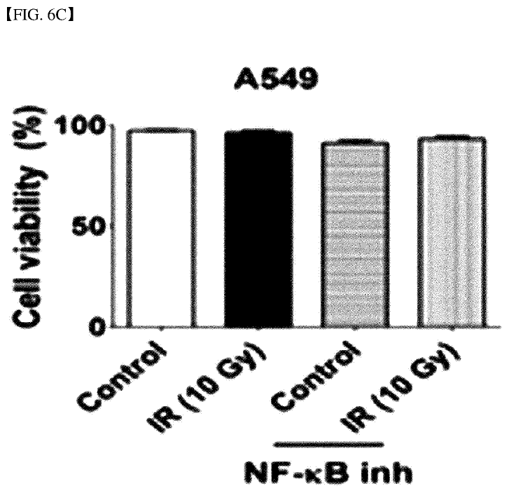

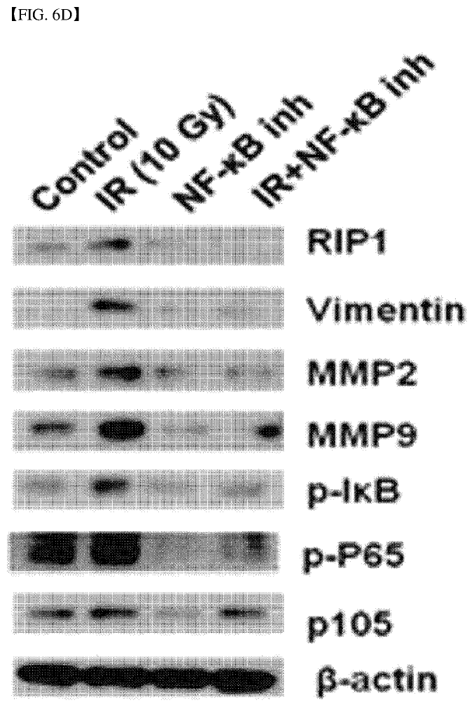

Lung cancer cell line A549 (control) and IR-treated lung cancer cell line A549 (IR) were placed in an upper chamber by 5×10⁴ in a Matrigel-coated transwell and treated with an NF-κB inhibitor. Then, 10% FBS medium was placed in the lower chamber, and cells passing through the transwell were stained after 24 hours to confirm cell invasion, and the results are shown in FIGS. 6A and 6B.

As shown in FIGS. 6A and 6B, changes in cell invasion and cell migration induced by treatment with NF-κB inhibitor were confirmed by performing invasion and migration analysis, and EMT marker expression was observed by performing Western blot. As a result, tt was confirmed that cell invasion and cell migration were reduced and RIP1 expression decreased when the IR-treated lung cancer cell line was treated with the NF-κB inhibitor. Meanwhile, as shown in FIG. 6C, it was confirmed that the cell viability was almost similar to that of the lung cancer cell line (control) that was not treated with radiation.

In addition, lung cancer cell line A549 (control) and IR-treated lung cancer cell line A549 (IR) were seeded with 1×10⁶ in a 60π dish and were treated with an NF-κB inhibitor. The cells were harvested 24 hours later, and protein was extracted. Then, Western blot was performed to confirm the expressions of RIP1, vimentin, MMP2, MMP9, p-IκBα, p-P65, p50, and p105. The results are shown in FIG. 6D. As shown in FIG. 6D, it was confirmed that inhibition of NF-κB reduced cell metastasis and invasion and was associated with decreased expressions of RIP1 and related proteins.

### Example 7: Analysis of Correlation Between Inflammatory Response Activation and NF-Kb in IR-Treated Lung Cancer Cell Line

It is known that RIP1 not only functions as a kinase but is also involved in the inflammatory response. According to recent studies, it has been reported that the inflammatory response is related to the proliferation and metastasis of cancer cells. In the present invention, the relationship between inflammatory response activation and NF-κB in the IR-treated lung cancer cell line was confirmed.

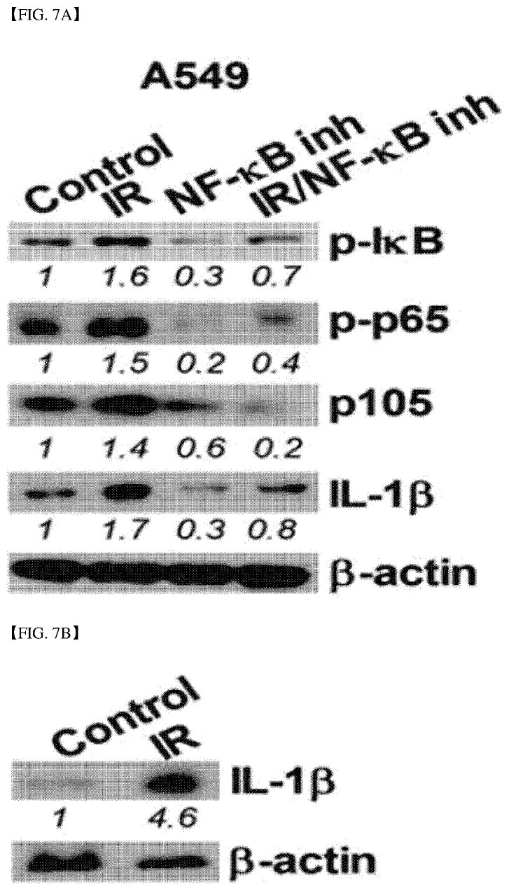

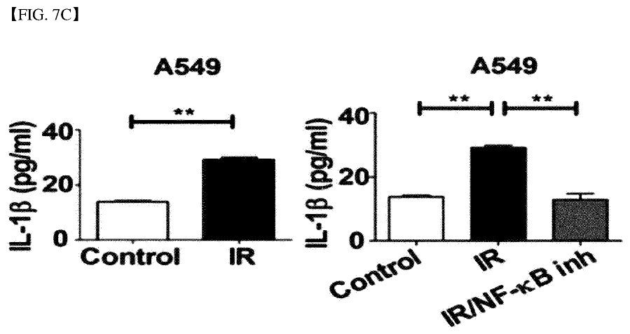

Lung cancer cell line A549 (control) and IR-treated lung cancer cell line A549 (IR) were seeded with 1×10⁶ in a 60π dish and treated with an NF-κB inhibitor. The cells were harvested 24 hours later, and protein was extracted. Then, Western blot was performed to confirm the expressions of p-IκBα, p-P65, p105, IL-1β, and 1β-action. The results are shown in FIG. 7A.

In addition, lung cancer cell line A549 (control) and IR-treated lung cancer cell line A549 (IR) were seeded with 1×10⁶ in a 60π dish. The cells were harvested 24 hours later, and protein was extracted. Then, Western blot was performed to confirm the expressions of IL-1β and β-action. The results are shown in FIG. 7B.

In addition, lung cancer cell line A549 (control) and the IR-treated lung cancer cell line A549 (IR) were placed in an upper chamber by 5×10⁴ in a Matrigel-coated transwell and treated with an NF-κB inhibitor (IR/NF-κB inh). Quantitative amounts of the expressed IL-1β were analyzed using their activated IL-1β protein antibody, and the results are shown in FIG. 7C.

As shown in FIGS. 7A, 7B, and 7C, it was confirmed that IL-1β (Interleukin 1β, a representative marker of the inflammatory response, was increased by IR treatment, and IL-1β expression was decreased when treated with an NF-κB inhibitor.

These results confirmed that the expression of IL-1β, one of the inflammatory response genes, was increased by IR treatment in lung cancer cell lines. In addition, the confirmed results indicated that the increase in the expression of IL-1β was related to the increase in the expression of genes related to cell migration and inflammatory response by RIP1.

In addition, it was confirmed that the increase in RIP1 by IR treatment promoted IL-1β expression and activation of NF-κB, its upper protein and that it induced EMT and cancer migration and invasion.

### Example 8. Effects of RIP1 on NF-κB Activation Mechanism and Cell Migration by IL-1Ra Treatment

It was confirmed that IR-induced IL-1β promoted cancer cell migration and invasion via EMT induction.

Specifically, considering that IR-induced up-regulation of NF-κB and RIP1 promoted cancer cell migration and invasion, it was confirmed whether IR-induced up-regulation of IL-1β could affect cancer cell migration by IR.

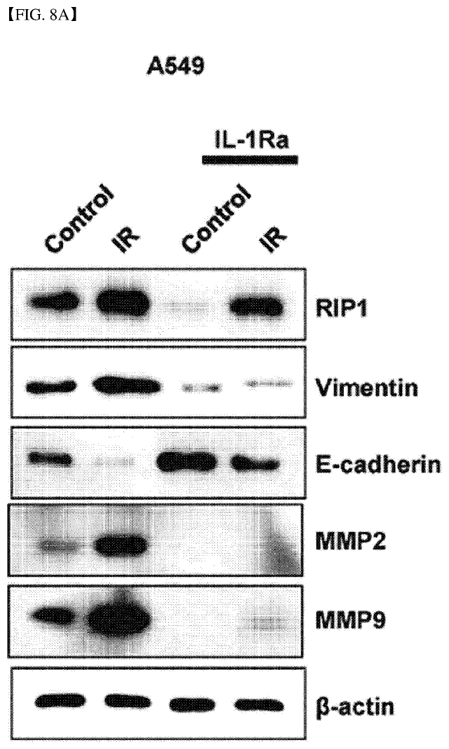

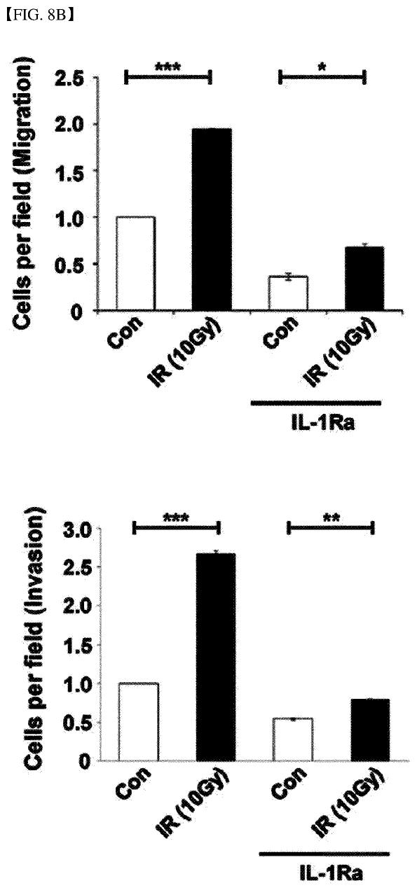

Immunoblot analysis of samples treated with or without IL-Ra (an antagonist of IL-1β) under IR irradiation conditions was performed, and the results are shown in FIG. 8A.

As shown in FIG. 8A, it was confirmed that IR showed an increase of vimentin and MMP-2/9 expressions as well as a decrease of E-cadherin. However, it was confirmed that the treatment of IL-Ra could exhibit a therapeutic effect by inhibiting this action in the underlying mechanism of RIP.

In addition, as shown in FIG. 8B, IR treatment increased the migration/invasion of A549 cells by more than 2-fold, and this action was almost completely blocked by IL-Ra. These results demonstrate that IL-1β might be a critical novel component involved in IR-induced migration/invasion

### Example 9. Expressions of IR Induced IL-1β and its Receptors, IL-1RI/II, In Vitro and In Vivo

It was confirmed whether IR changed the expression level of IL-1RI/II.

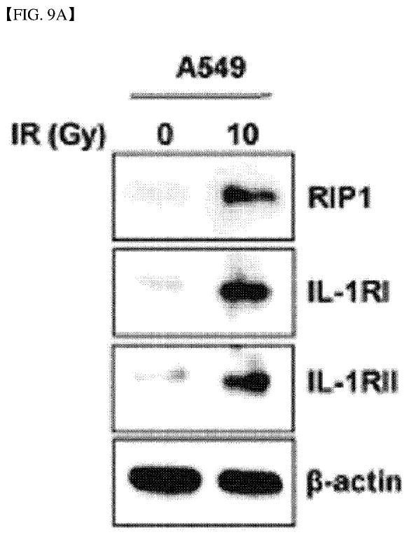

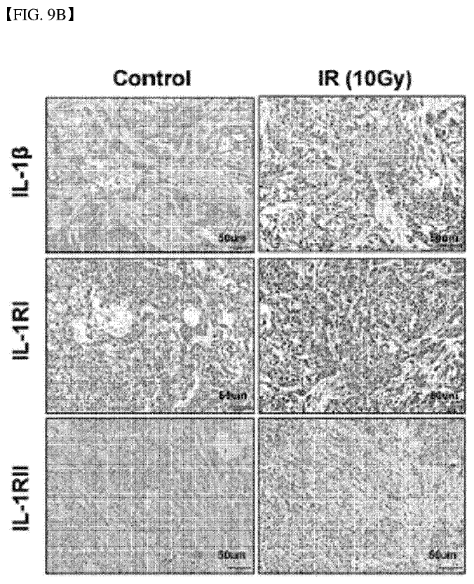

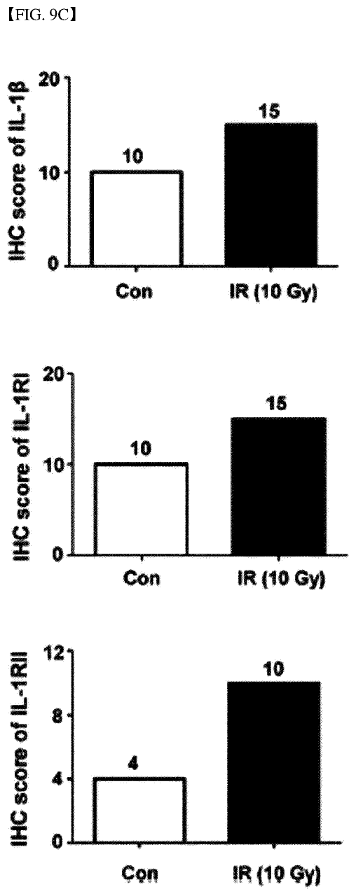

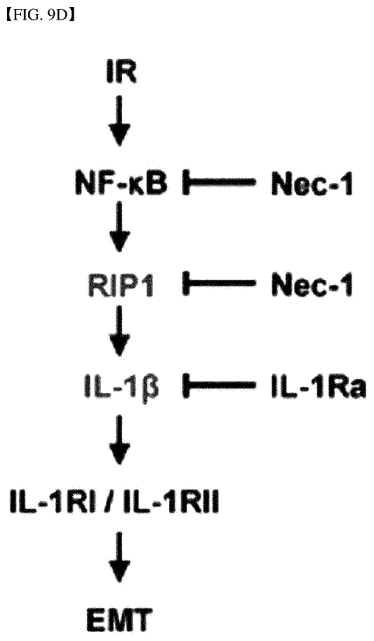

As shown in FIGS. 9A and 9B, 10 Gy IR treatment induced IL-1RI/II in vitro and in vivo. Specifically, immunoblotting analyses with cell lysates in vitro demonstrated that IR treatment increased the levels of both IL-1RI and II

In addition, to evaluate the induction of IL-1RI/II in vivo, A549 cells were subcutaneously injected into nude mice and allowed to develop to xenografts, which were then exposed to IR (10 Gy). Mice were sacrificed after 48 hours, and xenograft tissues were collected for immunohistochemical (IHC) analysis. The IHC data showed that the expression levels of IL-1b and its receptors, IL-1RI/II, were upregulated in IR-treated xenograft tissues. The scoring of the above results is shown in FIG. 9C. Quantitative analysis of the IHC results showed that IL-1β and IL-1RI/II were upregulated more than 2-fold in IR-treated tissues compared to non IR-treated xenografts tissues.

As shown in FIG. 9D, these results suggest that IR might induce cancer cell migration and invasion via activating the new signaling pathway of NF-κβ-RIP1-IL-1β-IL1RI/II-EMT.

As can be seen above, the present invention confirmed that metastasis of lung cancer is increased by radiotherapy, and RIP1 and EGFR, NF-κB, STAT-3, Src, IL-1RI, IL-1RII, and IL-1β signaling system is involved in the increase in metastasis. In addition, it was confirmed that RIP1 is inhibited during radiotherapy, thereby inhibiting cancer metastasis and invasion ability to eliminate the side effects caused by radiotherapy.

From the above description, those skilled in the art to which the present invention pertains will understand that the present invention may be embodied in other specific forms without changing the technical spirit or essential characteristics thereof. In this regard, it should be understood that the embodiments described above are illustrative in all respects and not restrictive. For the scope of the present invention, it should be construed that rather than the above detailed description, all changes or modifications derived from the meaning and scope of the following claims and their equivalents are included in the scope of the present invention.

1. A method for providing information for diagnosis of metastasis of radiotherapy-treated lung cancer, the method comprising steps of:
(a) measuring an expression level of receptor-interacting protein kinase 1 (RIP1) in a sample from a lung cancer patient who has undergone radiotherapy;
(b) measuring an expression level of RIP1 in a normal control sample; and
(c) comparing the expression levels of step (a) and step (b).

2. The method of claim 1, further comprising
(d) determining that a metastatic potential of radiotherapy-treated lung cancer is high when the expression level of step (a) is equal to or greater than the expression level of step (b).

3. The method of claim 1, wherein the measuring of an expression level of RIP1 is to measure an amount of protein or mRNA.

4. The method of claim 1, wherein step (a) further includes a step of measuring an expression level of any one or more selected from the group consisting of EGFR, NF-κB, STAT-3, Src, IL-1RI, IL-1RII, and IL-113,
wherein step (b) further includes a step of measuring an expression level of any one or more selected from the group consisting of EGFR, NF-κB, STAT-3, Src, IL-1RI, IL-1RII, and IL-1β from the normal control sample, and
wherein step (c) further includes a step of comparing the expression levels of step (a) and step (b) measured in step (a) and step (b), respectively.

5. The method of claim 4, wherein the measuring of the expression levels of steps (a) and (b) is to measure all of EGFR, NF-κB, STAT-3, Src, IL-1RI, IL-1RII, and IL-1β.

6. A kit for diagnosing lung cancer metastasis induced by radiation, the kit comprising an agent for measuring an expression level of RIP1.

7. The kit of claim 6, further comprising an agent for measuring an expression level of any one or more selected from the group consisting of EGFR, NF-κB, STAT-3, Src, IL-1RI, IL-1RII, and IL-1β.

8. A method for inhibiting metastasis of radiotherapy-treated lung cancer, the method comprising steps of:
(a) measuring an expression level of receptor-interacting protein kinase 1 (RIP1) in a sample from a lung cancer patient who has undergone radiotherapy;
(b) measuring an expression level of RIP1 in a normal control sample;
(c) comparing the expression levels of step (a) and step (b); and
(d) when the expression level of step (a) is higher than the expression level of step (b), administering a therapeutic agent for lung cancer or a metastasis inhibitor in a therapeutically effective amount.

9. The method of claim 8, wherein step (a) further includes a step of measuring an expression level of any one or more selected from the group consisting of EGFR, NF-κB, STAT-3, Src, IL-1RI, IL-1RII, and IL-1β,
wherein step (b) further includes a step of measuring an expression level of any one or more selected from the group consisting of EGFR, NF-κB, STAT-3, Src, IL-1RI, IL-1RII, and IL-1β from the normal control sample, and
wherein step (c) further includes a step of comparing the expression levels of step (a) and step (b) measured in step (a) and step (b), respectively.
